(19) United States
(12) Patent Application Publication
(10) Pub. No.: US 2026/0272619 A1
(43) Pub. Date: Sep. 17, 2026

(54) DENTAL MODELING DEVICE AND METHOD

(71) Applicant: ZETTA25 AG, Zurich (CH)

(72) Inventors: Hong Keun Kim, Oberursel (DE); Anders Adamson, Darmstadt (SE)

(21) Appl. No.: 19/081,602

(22) Filed: Mar. 17, 2025

(51) Int. Cl. A61C 9/00; A61B 1/00; A61B 1/24; A61C 13/34; G06T 17/00; G06T 19/20

(52) U.S. Cl. CPC A61C 9/0053; A61B 1/00172; A61B 1/24; A61C 13/34; G06T 17/00; G06T 19/20; G06T 2200/24; G06T 2210/41; G06T 2219/2016

(57) ABSTRACT

Various embodiments disclosed relate to imaging and analysis of a patient's dental arch. A 3D model and a selection of correlated 2D images are presented to a practitioner. One or more of the 2D images include highlighted borders to alert the practitioner when an alternate image can be selected to improve information concerning a feature of interest.

### TECHNICAL FIELD

Examples disclosed herein generally relate to dental analysis tools and dental modelling.

### BACKGROUND

Imaging tools are useful in dental treatment. Accurate representation of teeth and other features associated with a dental arch is important to provide the best analysis and diagnosis. Improved imaging and characterization and improved user interfaces are desired for dental analysis.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1A illustrates a display of a user device including a dental arch in accordance with some example embodiments.

FIG. 1B illustrates a system diagram concerning information about a dental arch in accordance with some example embodiments.

FIG. 2A illustrates an operation of collecting data concerning a dental arch in accordance with some example embodiments.

FIG. 2B illustrates another operation of collecting data concerning a dental arch in accordance with some example embodiments.

FIG. 3A illustrates a plurality of 2D images correlated to a dental arch in accordance with some example embodiments.

FIG. 3B illustrates a 3D image of a dental arch in accordance with some example embodiments.

FIG. 4A illustrates another plurality of 2D images correlated to a dental arch in accordance with some example embodiments.

FIG. 4B illustrates a selected 2D image from FIG. 4A in accordance with some example embodiments.

FIG. 5 illustrates a flow diagram of a method of analyzing a dental arch in accordance with some example embodiments.

FIG. 6 illustrates generally an example of a block diagram of a machine upon which any one or more of the techniques discussed herein may perform in accordance with some embodiments.

### DETAILED DESCRIPTION

In the following detailed description, reference is made to the accompanying drawings which form a part hereof, and in which is shown, by way of illustration, specific embodiments in which the invention may be practiced. In the drawings, like numerals describe substantially similar components throughout the several views. These embodiments are described in sufficient detail to enable those skilled in the art to practice the invention. Other embodiments may be utilized and structural, or logical changes, etc. may be made without departing from the scope of the present invention.

The following, non-limiting examples, detail certain aspects of the present subject matter to solve the challenges and provide the benefits discussed herein, among others.

An example of subject matter (e.g., a computer-implemented method) may comprise collecting data concerning a patient's dental arch, including 3D spatial data of a portion of the patient's dental arch and a plurality of 2D images with a correlation to the 3D spatial data, constructing a 3D model of a portion of the patient's dental arch using processing circuitry, mapping the plurality of 2D images to the 3D model using the correlation, and presenting a user interface including a mosaic of multiple 2D images from the plurality of 2D images and presenting highlighted borders of the multiple 2D images in the mosaic of multiple 2D images.

In an example, which may be combined with any one or more examples described herein, collecting data concerning a patient's dental arch optionally includes collecting a plurality of 2D images using a single image capture for each of the plurality of 2D images.

In an example, which may be combined with any one or more examples described herein, collecting data concerning a patient's dental arch optionally includes collecting a plurality of 2D images using one of a visible light spectrum, a near infrared light spectrum, or an ultraviolet light spectrum.

In an example, which may be combined with any one or more examples described herein, the subject matter optionally includes presenting a control to the user to rotate the 3D model and to present different 2D images corresponding to a variable viewpoint of the 3D model.

In an example, which may be combined with any one or more examples described herein, the subject matter optionally includes presenting an option to select a region of interest near a first border in a first 2D image and presenting an option to change to a second 2D image to display the region of interest within a second border of the second 2D image.

In an example, which may be combined with any one or more examples described herein, the subject matter optionally includes presenting an option to select a region of interest near a first border in a first 2D image and automatically changing to a second 2D image to display the region of interest within a second border of the second 2D image.

In an example, which may be combined with any one or more examples described herein, the subject matter optionally includes presenting an option to switch a display between the 3D model and a mosaic of multiple 2D images at a selected viewing orientation.

An example of subject matter (e.g., at least one non-transitory machine-readable medium including instructions) may comprise instructions which when executed by processing circuitry cause the processing circuitry to perform operations to collect data concerning a patient's dental arch including 3D spatial data of a portion of the patient's dental arch and a plurality of 2D images with a correlation to the 3D spatial data, construct a 3D model of a portion of the patient's dental arch, map the plurality of 2D images to the 3D model using the correlation, and present a user interface including a mosaic of multiple 2D images from the plurality of 2D images and present highlighted borders of the multiple 2D images in the mosaic of multiple 2D images.

In an example, which may be combined with any one or more examples described herein, the processing circuitry to perform operations is optionally configured to collect a plurality of 2D images using a single image capture for each of the plurality of 2D images.

In an example, which may be combined with any one or more examples described herein, the processing circuitry to perform operations is optionally configured to collect a plurality of 2D images using one of a visible light spectrum, a near infrared light spectrum, or an ultraviolet light spectrum.

In an example, which may be combined with any one or more examples described herein, the processing circuitry to perform operations is optionally configured to present a control to the user to rotate the 3D model and to present different 2D images corresponding to a variable viewpoint of the 3D model.

In an example, which may be combined with any one or more examples described herein, the processing circuitry to perform operations is optionally configured to present an option to select a region of interest near a first border in a first 2D image and present an option to change to a second 2D image to display the region of interest within a second border of the second 2D image.

In an example, which may be combined with any one or more examples described herein, the processing circuitry to perform operations is optionally configured to present an option to select a region of interest near a first border in a first 2D image and automatically change to a second 2D image to display the region of interest within a second border of the second 2D image.

In an example, which may be combined with any one or more examples described herein, the processing circuitry to perform operations is optionally configured to present an option to switch a display between the 3D model and a mosaic of multiple 2D images at a selected viewing orientation.

An example of subject matter (e.g., a dental imaging system) may comprise a scanner configured to collect data concerning a patient's dental arch including 3D spatial data of a portion of the patient's dental arch and a plurality of 2D images with a correlation to the 3D spatial data, processing circuitry to construct a 3D model of a portion of the patient's dental arch and map the plurality of 2D images to the 3D model using the correlation, and a user interface to present the 3D model and the plurality of 2D images, the user interface configured to present a mosaic of multiple 2D images from the plurality of 2D images and present highlighted borders of the multiple 2D images in the mosaic of multiple 2D images.

In an example, which may be combined with any one or more examples described herein, the user interface optionally includes a toggle configured to switch between the mosaic of multiple 2D images and the 3D model and a selection control to choose from different 2D images of the plurality of 2D images.

In an example, a system, method, or apparatus may optionally combine any portion or combination of any portion of any one or more of the examples described herein.

The following detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

The systems and techniques described herein may be used to receive data from imaging devices that are used to capture data about a patient's dental arch. The systems and techniques described herein may be used to present accurate color and texture information to a practitioner to aid in diagnosis and treatment of dental conditions.

In constructing a 3D model of a patient's dental arch, 2D images can be taken in addition to topographic data. Information from images can be useful in diagnosis and treatment of dental conditions. It is important to obtain accurate and consistent image data across all of the patient's dental arch. Because multiple images are often obtained from several different imaging device viewpoints or orientations, it can be difficult to obtain accurate and consistent information from multiple images. An average color or average texture can be misleading and less accurate than a given single image.

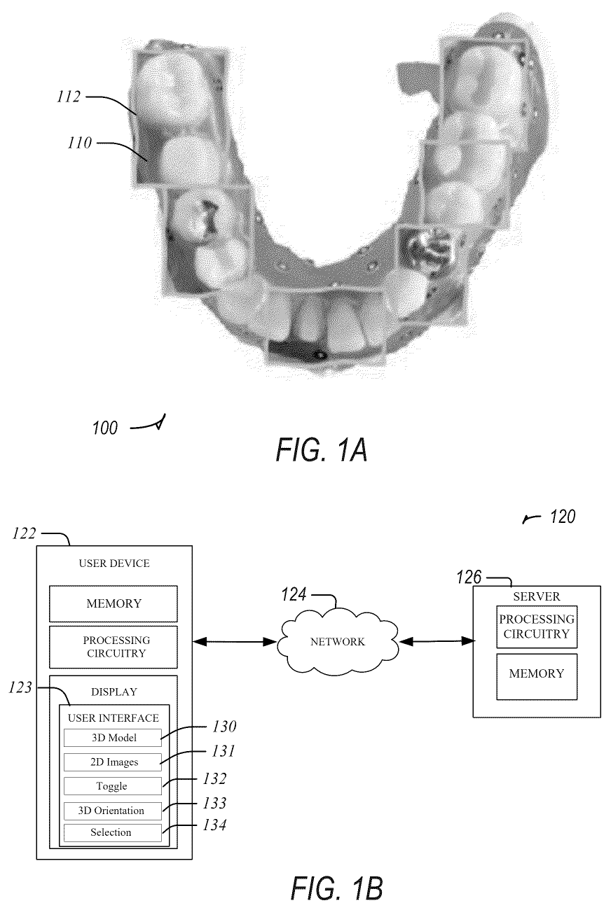

FIG. 1A shows a displayed dental arch 100 according to one example. The displayed dental arch 100 includes multiple 2D images 110. In one example, a scanner is used to collect the multiple 2D images 110. Examples of scanners include, but are not limited to, hand-held scanning devices that are moved around a patient's mouth with a scanning window oriented by a practitioner to capture the multiple 2D images 110. Although selected individual images 110 are shown in FIG. 1A, many additional images in regions that overlap the shown images 110 are also captured, but not shown in FIG. 1A. Other images in different locations are optionally viewed as selected by a practitioner, as discussed in more detail below. Given that multiple imaging angles are used to collect data for the dental arch 100, multiple images are often needed to fully record a condition in all locations of the dental arch 100. The displayed dental arch 100 further includes borders 112 for the multiple 2D images 110. In the example of FIG. 1A, the borders 112 are highlighted to aid a practitioner, and to alert the practitioner when an area of interest is at or near a border 112 in a group of multiple 2D images 110.

When single images 110 are viewed, without any averaging of color, texture, or other image pixel properties, the data is more reliable. For example, a color in a specific location within an image 110 is more realistic than if colors from multiple images are averaged in to a combined image. Other information from a given image can also be more accurate in specific locations when obtained from a single image, or a smaller number of images local to the point of interest. Examples of other information, include, but are not limited to, topography, texture, pattern, surface roughness, etc.

In viewing the displayed dental arch 100, it can be useful for a practitioner to know where a border of each of the multiple 2D images 110 lies in relation to any given image 110. In practice, if a feature of interest lies on or near a border 112, then a different image can be selected, such that the feature of interest is more centered within a given image, or at least farther from a border 112, to ensure that the data of the image being viewed regarding the feature of interest is more accurate, and shown within better context of surrounding areas. Although the dental arch 100 of FIG. 1A, shows only images 110 that are adjacent to one another, and not overlapping, a collection of images that relate to the dental arch 100 may include several images 110 that if displayed at the same time, would include overlapping images 110. As discussed in more detail below, only some of a number of collected 2D images may be shown at one time to a practitioner, even thought other images are available, but not displayed at the time.

In the example of FIG. 1A, one or more borders 112 are highlighted to better aid the practitioner in knowing when a feature of interest is located near a border 112. Examples of highlighting include, but are not limited to, displaying a border color, displaying a border line with an identifiable thickness, displaying a border line pattern, etc.

FIG. 1B illustrates a system diagram 120 concerning information about a dental arch, such as the arch 100 illustrated in FIG. 1A. The system diagram 120 illustrates a user device 122 in communication with a server 126 via a network 124 (e.g., the Internet). Examples of user devices 122 include, but are not limited to, laptop computers, desktop computers, tablets, etc. In one example, the user device 122 may include processing circuitry and memory to collect data concerning a patient's dental ach, and to generate images and/or a 3D model of the patient's dental arch. In selected examples, functions concerning analyzing the patient's dental arch may take place at the user device 122, without any functions taking place at the server 126. In other examples, one or more functions concerning analyzing the patient's dental arch may take place at the server 126. For example, the server 126 may store data concerning the patient's dental arch. In one example, user device 122 may store data concerning the patient's dental arch. In one example, the server 126 may construct, using processing circuitry, a 3D model of all or a portion of the patient's dental arch.

After one or more data collection functions and processing functions are performed, a user interface 123 is generated at the user device 122. The user interface 123 may include a 3D model 130 of a dental arch similar to dental arch 100 from FIG. 1A. The user interface 123 may include a plurality of 2D images 131 of a dental arch similar to dental arch 100 from FIG. 1A. In one example, the plurality of 2D images include a correlation to the 3D model. In one example, the user interface 123 includes a toggle 132 to switch between a view of the 3D model 130 and the plurality of 2D images 131. In one example, the user interface 123 includes a control 133 that is used to orient the 3D model 130. Examples of orientation include, but are not limited to rotation about one or more axes, translation, zooming in or zooming out, etc. In one example, the user interface 123 includes a selection control 134 to choose from different 2D images, or different sets of 2D images.

A correlation between the 3D data and the 2D data is used to, for example, give a 3D point the correct color information obtained by the 2D data (e.g., photo). One way to correlate 2D and 3D data is through calibration. This can be achieved by the simultaneous acquisition of a calibration object that has structures with known geometry and color, such as a calibration plate with a dotted pattern. The simultaneous acquisition allows mapping the information from a 2D sensor to a 3D data point. However, the detailed method of correlation depends on the underlying optical principle. If the 3D and 2D data are acquired by the same sensor, then no calibration might be needed for 3D to 2D mapping. In selected examples where multiple sensors are utilized, calibration may be used, involving techniques such as look-up tables and coordinate transforms for the applied camera model and used optics.

In one example operation, using the system 120, a practitioner is able to rotate or otherwise change a view of a generated 3D model to a view of interest. The practitioner is then able to display a mosaic of 2D images related to a selected view of the 3D model. By displaying highlighted borders of images from the mosaic of 2D images, the practitioner is able to decide if a feature of interest is sufficiently within a given 2D image. If a different 2D image is desired, for example, to show a feature of interest that lies in a more centered location within a 2D image, the practitioner may select a different 2D image, or a different set of 2D images to obtain better detail and/or better surrounding context for the feature of interest. A process of selecting a different 2D image is discussed in more detail below.

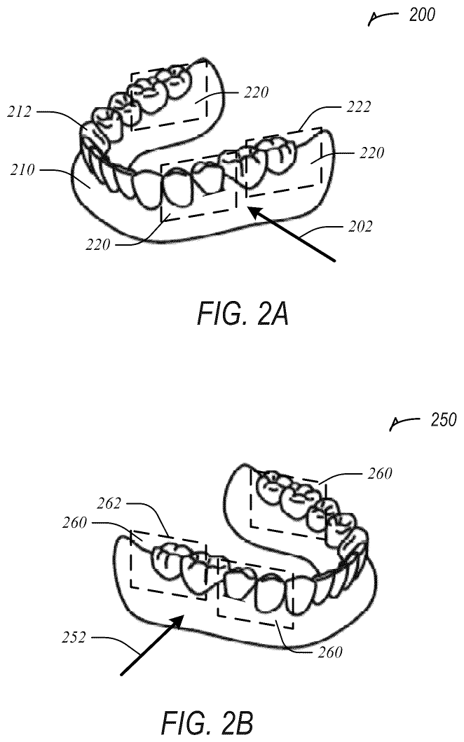

FIGS. 2A and 2B show selected details of collecting data concerning a patient's dental arch. In FIG. 2A, a patient's dental arch 210 is imaged. The dental arch 210 includes a number of individual teeth 212. In the stage 200 shown in FIG. 2A, a number of individual first images 220 are generated from a first direction 202. One or more of the first images 220 include information about borders 222 of the first images 220. In the stage 250 shown in FIG. 2B, a number of second images 260 are generated from a second direction 252. One or more of the second images 260 include information about borders 262 of the second images 260. The examples of FIGS. 2A and 2B show only two different directions to record images. In practice a large range of different orientations are used to fully characterize a dental arch 210. Additionally, FIGS. 2A and 2B only show a small number of images 220, 260 that do not overlap. In one example several overlapping images 220, 260 are obtained. In one example, a system, such as system 120, sorts through available images and displays them to a practitioner in manageable sets of images for a given view. In the examples shown, only non-overlapping images are shown, with borders that are highlighted as discussed above. Other sets of images may be selected for a given view in other examples.

A number of different imaging or scanning techniques may be utilized to collect data. In one example, visible light images are used to capture images 220, 260. In one example, near infrared light images are used to capture images 220, 260. In one example, ultraviolet light images are used to capture images 220, 260. When illuminated with near infrared light, ultraviolet or blue light information relevant for diagnostics can be made visible such as cariogenic tooth tissue that exhibits fluorescence (in the case of ultraviolet or blue light). In one example, texturing or topography images are used to capture images 220, 260.

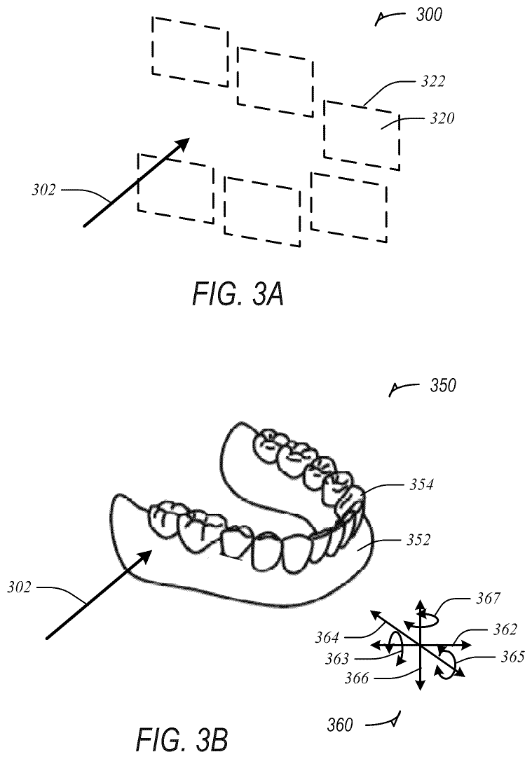

FIG. 3A shows a mosaic 300 of multiple 2D images 320 for a selected view direction 302. Similar to examples discussed above, the multiple 2D images 320 are displayed along with highlighted borders 322. FIG. 3B shows a 3D model 350 of a patient's dental arch that is formed with 3D spatial data. One example of 3D spatial data includes interferometry scanning. Other forms of 3D spatial data to construct a 3D model are also within the scope of the invention. The mosaic 300 of multiple 2D images 320 from FIG. 3A are correlated to the 3D model 350 from FIG. 3B. Because of the correlation between the mosaic 300 of multiple 2D images 320 and the 3D model 350, a selected rotation or translation view of the 3D model 350 will correlate to a corresponding mosaic 300 of multiple 2D images 320 taken along the same direction. In FIGS. 3A and 3B, the common direction 302 is a common correlation between FIGS. 3A and 3B. Other correlations include locating a given image 320 over a corresponding portion of the 3D model 350.

FIG. 3B further shows a set of orientation options 360. Rotation 363 is possible about X-axis 362. Translation is also possible back and forth along X-axis 362. Rotation 365 is possible about Z-axis 364. Translation is also possible back and forth along Z-axis 364. Rotation 367 is possible about Y-axis 366. Translation is also possible back and forth along Y-axis 366. In one example, a toggle, such as toggle 132 from user interface 123 of FIG. 1B switches between the 3D model 350 and the corresponding mosaic 300 of multiple 2D images 320.

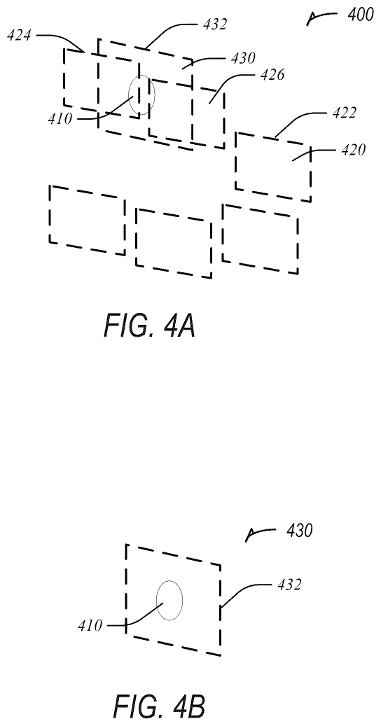

FIGS. 4A and 4B show an example method for analyzing a dental arch utilizing borders of images and switching images for improved analysis. In FIG. 4A, a mosaic 400 of 2D images 420 are shown, similar to other examples discussed above. One or more of the 2D images 420 are shown with a border 422 highlighted to make a practitioner aware of an extent of each individual image 420. As discussed above, when colors or textures, or other properties of multiple images are averaged, detail specific to a given area is lost. In many instances, it is desirable to keep individual detail specific to each specific region of a dental arch. In one example, each individual 2D image 420 includes detail from a single image capture. In this way, color, texture, etc. is preserved for each image, and is not averaged over multiple or all images. In another example, each individual 2D image 420 includes detail only averaged from a local region of the dental arch, and is not averaged over the entire dental arch.

As noted, 2D images include detail to alert a practitioner where borders 422 are for a given image. The borders 422 are highlighted, for example by including a high visibility color in a user interface on a user device, or other type of highlighting. FIG. 4A shows a feature of interest 410 that lies adjacent to borders 422 or spans two different 2D images 420 in the mosaic 400. It is desirable to both preserve local detail of an individual 2D image 420, and to be able to view accurate context within a single image around the feature of interest 410. Because the borders 422 are highlighted, a practitioner is aware that adjacent images 424 and 426 have borders 422 that lie across the feature of interest 410.

In one example, a practitioner is able to switch one or more images 420 to select an alternative image that fully encompasses the feature of interest 410. In one example, a user interface, such as user interface 123 provides a selection control 134 to change a selection of an individual image, or between different mosaics of images. An alternative image 430 is shown in FIG. 4A that encompasses the feature of interest 410 completely within its border 432. FIG. 4B shows the alternative image 430 by itself with the feature of interest 410 completely within its border 432. In other examples, the alternative image 430 is an improved location that encompasses more of the feature of interest 410 within its borders 432 than 2D images 420 in the mosaic 400.

Selection of an alternative image 430 may be performed manually by the practitioner. In other examples, an alternative image 430 may be automatically selected after a practitioner indicates where the feature of interest 410 is located. For example, as a practitioner zooms in on the feature of interest 410, the user interface may automatically select an alternative image 430 that improves a location of the feature of interest 410 within borders 432. Zooming may not necessarily be needed. Once the practitioner indicates where the feature of interest 410 is located, the user interface may automatically select an alternative image 430 that improves a location of the feature of interest 410 within borders 432.

Examples of features of interest include, but are not limited to, a margin of a crown, a retraction cord, textured caries information, etc. In one example, a practitioner performing the analysis is not located in the same physical location as the patient. For example, in remote medicine, a practitioner may be located in another part of the country, and may be diagnosing multiple patients in a time shifted manner, not at the same time that a patient dental arch is being imaged. In situations such as these, it is desirable to have images as accurate as possible to provide the best diagnosis. By providing highlighted borders of individual images, a practitioner working remotely or at a different time can more accurately find alternate images as needed to provide detail of a specific feature of interest, without having to interact with a patient in real time.

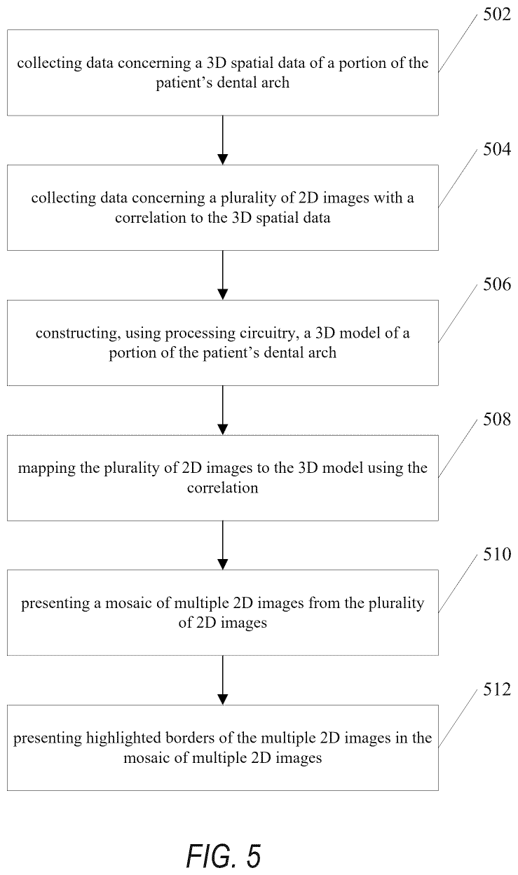

FIG. 5 shows a flow diagram of an example method of modelling a dental arch. In operation 502, data concerning a 3D spatial data of a portion of the patient's dental arch is collected. In operation 504, data concerning a plurality of 2D images with a correlation to the 3D spatial data is collected. In operation 506, a 3D model of a portion of the patient's dental arch is constructed, using processing circuitry. In operation 508, the plurality of 2D images are mapped to the 3D model using the correlation. In operation 510, a user interface is presented, including a mosaic of multiple 2D images from the plurality of 2D images. In operation 512, highlighted borders of the multiple 2D images are presented in the mosaic of multiple 2D images.

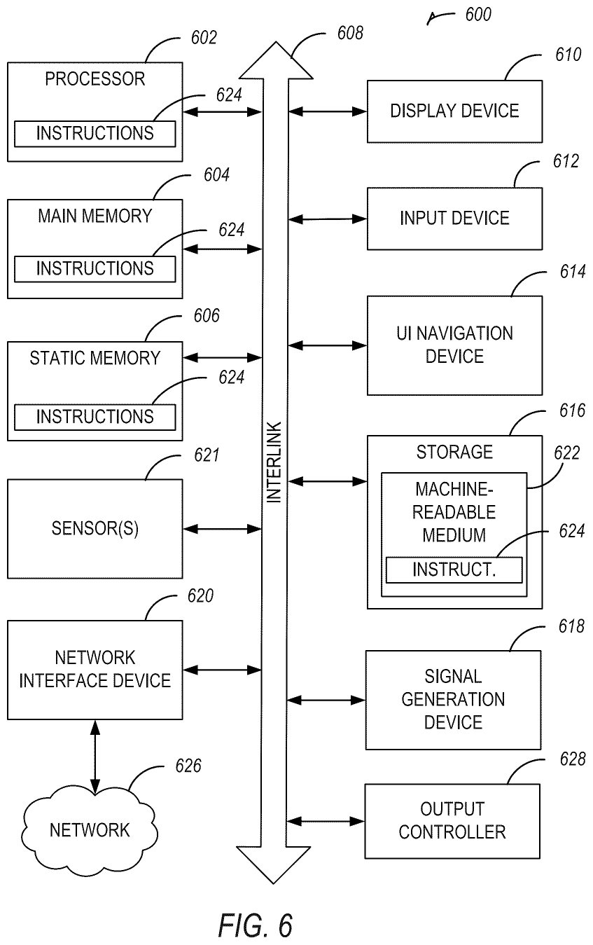

FIG. 6 illustrates generally an example of a block diagram of a machine 600 upon which any one or more of the techniques (e.g., methodologies) discussed herein may perform in accordance with some embodiments. In alternative embodiments, the machine 600 may operate as a standalone device or may be connected (e.g., networked) to other machines. In a networked deployment, the machine 600 may operate in the capacity of a server machine, a client machine, or both in server-client network environments. In an example, the machine 600 may act as a peer machine in peer-to-peer (P2P) (or other distributed) network environment. The machine 600 may be a personal computer (PC), a tablet PC, a set-top box (STB), a personal digital assistant (PDA), a mobile telephone, a web appliance, a network router, switch or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein, such as cloud computing, software as a service (SaaS), other computer cluster configurations.

Examples, as described herein, may include, or may operate on, logic or a number of components, modules, or mechanisms. Modules are tangible entities (e.g., hardware) capable of performing specified operations when operating. A module includes hardware. In an example, the hardware may be specifically configured to carry out a specific operation (e.g., hardwired). In an example, the hardware may include configurable execution units (e.g., transistors, circuits, etc.) and a computer readable medium containing instructions, where the instructions configure the execution units to carry out a specific operation when in operation. The configuring may occur under the direction of the executions units or a loading mechanism. Accordingly, the execution units are communicatively coupled to the computer readable medium when the device is operating. In this example, the execution units may be a member of more than one module. For example, under operation, the execution units may be configured by a first set of instructions to implement a first module at one point in time and reconfigured by a second set of instructions to implement a second module.

Machine (e.g., computer system) 600 may include a hardware processor 602 (e.g., a central processing unit (CPU), a graphics processing unit (GPU), a hardware processor core, or any combination thereof), a main memory 604 and a static memory 606, some or all of which may communicate with each other via an interlink (e.g., bus) 608. The machine 600 may further include a display unit 610, an alphanumeric input device 612 (e.g., a keyboard), and a user interface (UI) navigation device 614 (e.g., a mouse). In an example, the display unit 610, alphanumeric input device 612 and UI navigation device 614 may be a touch screen display. The machine 600 may additionally include a storage device (e.g., drive unit) 616, a signal generation device 618 (e.g., a speaker), a network interface device 620, and one or more sensors 621, such as a global positioning system (GPS) sensor, compass, accelerometer, or other sensor. The machine 600 may include an output controller 628, such as a serial (e.g., universal serial bus (USB), parallel, or other wired or wireless (e.g., infrared (IR), near field communication (NFC), etc.) connection to communicate or control one or more peripheral devices (e.g., a printer, card reader, etc.).

The storage device 616 may include a machine readable medium 622 that is non-transitory on which is stored one or more sets of data structures or instructions 624 (e.g., software) embodying or utilized by any one or more of the techniques or functions described herein. The instructions 624 may also reside, completely or at least partially, within the main memory 604, within static memory 606, or within the hardware processor 602 during execution thereof by the machine 600. In an example, one or any combination of the hardware processor 602, the main memory 604, the static memory 606, or the storage device 616 may constitute machine readable media.

While the machine readable medium 622 is illustrated as a single medium, the term “machine readable medium” may include a single medium or multiple media (e.g., a centralized or distributed database, or associated caches and servers) configured to store the one or more instructions 624.

The term “machine readable medium” may include any medium that is capable of storing, encoding, or carrying instructions for execution by the machine 600 and that cause the machine 600 to perform any one or more of the techniques of the present disclosure, or that is capable of storing, encoding or carrying data structures used by or associated with such instructions. Non-limiting machine-readable medium examples may include solid-state memories, and optical and magnetic media. Specific examples of machine-readable media may include: non-volatile memory, such as semiconductor memory devices (e.g., Electrically Programmable Read-Only Memory (EPROM), Electrically Erasable Programmable Read-Only Memory (EEPROM)) and flash memory devices; magnetic disks, such as internal hard disks and removable disks; magneto-optical disks; and CD-ROM and DVD-ROM disks.

The instructions 624 may further be transmitted or received over a communications network 626 using a transmission medium via the network interface device 620 utilizing any one of a number of transfer protocols (e.g., frame relay, internet protocol (IP), transmission control protocol (TCP), user datagram protocol (UDP), hypertext transfer protocol (HTTP), etc.). Example communication networks may include a local area network (LAN), a wide area network (WAN), a packet data network (e.g., the Internet), mobile telephone networks (e.g., cellular networks), and wireless data networks (e.g., Institute of Electrical and Electronics Engineers (IEEE) 802.11 family of standards known as Wi-Fi®, IEEE 802.16 family of standards known as WiMax®), IEEE 802.15.4 family of standards, peer-to-peer (P2P) networks, among others. In an example, the network interface device 620 may include one or more physical jacks (e.g., Ethernet, coaxial, or phone jacks) or one or more antennas to connect to the communications network 626. In an example, the network interface device 620 may include a plurality of antennas to wirelessly communicate using at least one of single-input multiple-output (SIMO), multiple-input multiple-output (MIMO), or multiple-input single-output (MISO) techniques. The term “transmission medium” shall be taken to include any intangible medium that is capable of storing, encoding or carrying instructions for execution by the machine 600, and includes digital or analog communications signals or other intangible medium to facilitate communication of such software.

In the event of inconsistent usages between this document and any documents so incorporated by reference, the usage in this document controls. In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code can form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic disks, removable optical disks (e.g., compact disks and digital video disks), magnetic cassettes, memory cards or sticks, random access memories (RAMs), read only memories (ROMs), and the like.

The above description is intended to be illustrative, and not restrictive. For example, the above-described examples (or one or more aspects thereof) can be used in combination with each other. Other embodiments can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above Detailed Description, various features can be grouped together to streamline the disclosure. This should not be interpreted as intending that an unclaimed disclosed feature is essential to any claim. Rather, inventive subject matter can lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

What is claimed is:

1. A computer-implemented method comprising:
collecting data concerning a patient's dental arch, including:
3D spatial data of a portion of the patient's dental arch;
a plurality of 2D images with a correlation to the 3D spatial data;
constructing, using processing circuitry, a 3D model of a portion of the patient's dental arch;
mapping the plurality of 2D images to the 3D model using the correlation;
presenting a user interface, including:
presenting a mosaic of multiple 2D images from the plurality of 2D images; and
presenting highlighted borders of the multiple 2D images in the mosaic of multiple 2D images.

2. The method of claim 1, wherein collecting data concerning a patient's dental arch includes collecting a plurality of 2D images using a single image capture for each of the plurality of 2D images.

3. The method of claim 1, wherein collecting data concerning a patient's dental arch includes collecting a plurality of 2D images using a visible light spectrum.

4. The method of claim 1, wherein collecting data concerning a patient's dental arch includes collecting a plurality of 2D images using a near infrared light spectrum.

5. The method of claim 1, wherein collecting data concerning a patient's dental arch includes collecting a plurality of 2D images using an ultraviolet light spectrum.

6. The method of claim 1, further including presenting a control to the user to rotate the 3D model and to present different 2D images corresponding to a variable viewpoint of the 3D model.

7. The method of claim 1, further including presenting an option to select a region of interest near a first border in a first 2D image and presenting an option to change to a second 2D image to display the region of interest within a second border of the second 2D image.

8. The method of claim 1, further including presenting an option to select a region of interest near a first border in a first 2D image and automatically changing to a second 2D image to display the region of interest within a second border of the second 2D image.

9. The method of claim 1, further including presenting an option to switch a display between the 3D model and a mosaic of multiple 2D images at a selected viewing orientation.

10. At least one non-transitory machine-readable medium including instructions, which when executed by processing circuitry, causes the processing circuitry to perform operations to:
collect data concerning a patient's dental arch, including:
3D spatial data of a portion of the patient's dental arch;
a plurality of 2D images with a correlation to the 3D spatial data;
construct, using processing circuitry, a 3D model of a portion of the patient's dental arch;
map the plurality of 2D images to the 3D model using the correlation;
present a user interface, including:
present a mosaic of multiple 2D images from the plurality of 2D images; and
present highlighted borders of the multiple 2D images in the mosaic of multiple 2D images.

11. The at least one non-transitory machine-readable medium of claim 10, wherein the processing circuitry to perform operations is configured to collect a plurality of 2D images using a single image capture for each of the plurality of 2D images.

12. The at least one non-transitory machine-readable medium of claim 10, wherein the processing circuitry to perform operations is configured to collect a plurality of 2D images using a visible light spectrum.

13. The at least one non-transitory machine-readable medium of claim 10, wherein the processing circuitry to perform operations is configured to collect a plurality of 2D images using a near infrared light spectrum.

14. The at least one non-transitory machine-readable medium of claim 10, wherein the processing circuitry to perform operations is configured to collect a plurality of 2D images using an ultraviolet light spectrum.

15. The at least one non-transitory machine-readable medium of claim 10, wherein the processing circuitry to perform operations is further configured to present a control to the user to rotate the 3D model and to present different 2D images corresponding to a variable viewpoint of the 3D model.

16. The at least one non-transitory machine-readable medium of claim 10, wherein the processing circuitry to perform operations is further configured to present an option to select a region of interest near a first border in a first 2D image and presenting an option to change to a second 2D image to display the region of interest within a second border of the second 2D image.

17. The at least one non-transitory machine-readable medium of claim 10, wherein the processing circuitry to perform operations is further configured to present an option to select a region of interest near a first border in a first 2D image and automatically changing to a second 2D image to display the region of interest within a second border of the second 2D image.

18. The at least one non-transitory machine-readable medium of claim 10, wherein the processing circuitry to perform operations is further configured to present an option to switch a display between the 3D model and a mosaic of multiple 2D images at a selected viewing orientation.

19. A dental imaging system, comprising:
a scanner, configured to collect data concerning a patient's dental arch, including:
3D spatial data of a portion of the patient's dental arch;
a plurality of 2D images with a correlation to the 3D spatial data;
processing circuitry to construct a 3D model of a portion of the patient's dental arch;
processing circuitry to map the plurality of 2D images to the 3D model using the correlation;
a user interface to present the 3D model and the plurality of 2D images, the user interface configured to:
present a mosaic of multiple 2D images from the plurality of 2D images; and
present highlighted borders of the multiple 2D images in the mosaic of multiple 2D images.

20. The dental imaging system of claim 19, wherein the user interface further includes a toggle configured to switch between the mosaic of multiple 2D images and the 3D model.

21. The dental imaging system of claim 19, wherein the user interface further includes a selection control to choose from different 2D images of the plurality of 2D images.
